(19) United States
(12) Patent Application Publication
(10) Pub. No.: US 2023/0165546 A1
(43) Pub. Date: Jun. 1, 2023

(54) MEDICAL IMAGE PROCESSING APPARATUS, X-RAY DIAGNOSIS APPARATUS, AND NON-VOLATILE COMPUTER-READABLE STORAGE MEDIUM STORING THEREIN MEDICAL IMAGE PROCESSING PROGRAM

(71) Applicant: CANON MEDICAL SYSTEMS CORPORATION, Otawara-shi (JP)

(72) Inventors: Akihito TAKAHASHI, Nasushiobara (JP); Kenji MIZUTANI, Nasushiobara (JP); Mika TAKAYA, Nasushiobara (JP); Takahiro YAMAGUCHI, Otawara (JP)

(73) Assignee: CANON MEDICAL SYSTEMS CORPORATION, Otawara-shi (JP)

(21) Appl. No.: 18/057,437

(22) Filed: Nov. 21, 2022

(30) Foreign Application Priority Data
Nov. 30, 2021 (JP) 2021-193908

(51) Int. Cl. A61B 6/12; A61B 6/00

(52) U.S. Cl. CPC A61B 6/12; A61B 6/4441

(57) ABSTRACT

A medical image processing apparatus according to an embodiment includes processing circuitry that obtains first X-ray images, a second X-ray image taken later than the first X-ray images, first biological information to a periodic movement of a subject at the times of taking the first X-ray images and second biological information to a periodic movement of the subject at the time of taking the second X-ray image; detects a characteristic position to a first device from each of the first X-ray images; associates the characteristic position with a time phase in the first biological information, based on the first X-ray images and first biological information; and displays device information to the first device superimposed on the second X-ray image, by using a result of the associating and time phase in the second biological information.

### CROSS-REFERENCE TO RELATED APPLICATIONS

This application is based upon and claims the benefit of priority from Japanese Patent Application No. 2021-193908, filed on Nov. 30, 2021, the entire contents of which are incorporated herein by reference.

### FIELD

Embodiments described herein relate generally to a medical image processing apparatus, an X-ray diagnosis apparatus, and a non-volatile computer-readable storage medium storing therein a medical image processing program.

### BACKGROUND

Conventionally, a known X-ray diagnosis apparatus or image processing apparatus is configured, while treatment is carried out with reference to an X-ray image, to instantaneously display the X-ray image that ensures visibility of a treatment device. For example, when applying an image deformation to an X-ray image in a current frame, the X-ray diagnosis apparatus or the image processing apparatus may use a marker position in an X-ray image from a past frame corresponding to the same cardiac phase. A technique with this configuration is known by which, to alleviate difficulties in manipulations caused by temporal movements of a site of interest (e.g., a blood vessel site having a stenosis) due to pulsation of the heart or respiration, the site of interest keeps being displayed in a fixed position.

For example, a stent to be used in treatment of a blood vessel site having a stenosis is selected according to the length of the lesion having the stenosis. Further, when the lesion is longer than a stent, blood vessel treatment is carried out on a desired site by using a plurality of stents. On such occasion, if there is a gap between the plurality of stents, the risk of having a restenosis at the site between the stents may increase. For this reason, the manipulation of placing the stents in the lesion having a stenosis is performed carefully so that there is no gap between a first stent placed in advance and a second stent to be placed next.

However, in a cardiovascular region, the manipulation to place the second stent may have high difficulty, because the first stent placed in advance and the second stent to be placed next both move in X-ray images due to pulsation of the heart and respiration of the examined subject and/or because the first stent is difficult to see in the X-ray images due to the position of the treated site, the physique of the examined subject, or the like.

### BRIEF DESCRIPTION OF THE DRAWINGS

FIG. 1 is a block diagram illustrating an exemplary configuration of an X-ray diagnosis apparatus according to an embodiment;

FIG. 2 is a block diagram illustrating an exemplary configuration of a medical image processing apparatus according to the embodiment;

FIG. 3 is a flowchart illustrating an example of a procedure in a device display process according to the embodiment;

FIG. 4 is a drawing according to the embodiment illustrating an example of an X-ray image (a fluoroscopic image) before obtaining a plurality of first X-ray images;

FIG. 5 is a drawing according to the embodiment illustrating a display example in a frame at the time of an expansion of a first balloon moved to a blood vessel stenosis site;

FIG. 6 is a drawing according to the embodiment illustrating another display example in the frame at the time of the expansion of the first balloon moved to the blood vessel stenosis site;

FIG. 7 is a drawing according to the embodiment illustrating an example after the first balloon is removed subsequent to the expansion of the first balloon;

FIG. 8 is a drawing according to the embodiment illustrating examples of the positions of first markers (virtual markers) and end face positions (virtual end faces) of a first stent superimposed on a blood vessel region in a second X-ray image;

FIG. 9 is another drawing according to the embodiment illustrating examples of the positions of the first markers (the virtual markers) and the end face positions (the virtual end faces) of the first stent superimposed on the blood vessel region in the second X-ray image;

FIG. 10 is a drawing according to the embodiment illustrating an example of a second marker moved up to a virtual end face in a stenosis site; and

FIG. 11 is a drawing according to a first modification example of the embodiment illustrating an example in which the diaphragm is displayed in a first X-ray image.

### DETAILED DESCRIPTION

A medical image processing apparatus according to an embodiment includes processing circuitry. The processing circuitry is configured: to obtain a plurality of first X-ray images related to an examined subject and a second X-ray image related to the examined subject taken later than the plurality of first X-ray images; to obtain first biological information related to a periodic movement of the examined subject at the times of taking the plurality of first X-ray images and second biological information related to a periodic movement of the examined subject at the time of taking the second X-ray image; to detect the position of a characteristic part related to a first device from each of the plurality of first X-ray images; to associate the position of the characteristic part with a time phase in the first biological information, on the basis of the plurality of first X-ray images and the first biological information; and to cause a display to display device information related to the first device so as to be superimposed on the second X-ray image, by using a result of the associating process and a time phase in the second biological information.

Exemplary embodiments of a medical image processing apparatus, an X-ray diagnosis apparatus, and a medical image processing program will be explained below, with reference to the accompanying drawings. In the following embodiments, some of the elements referred to by using the same reference characters are assumed to perform the same operations, and duplicate explanations thereof will be omitted as appropriate. Further, the description of any of the embodiments is, in principle, similarly applicable to modification examples and the like.

### EMBODIMENTS

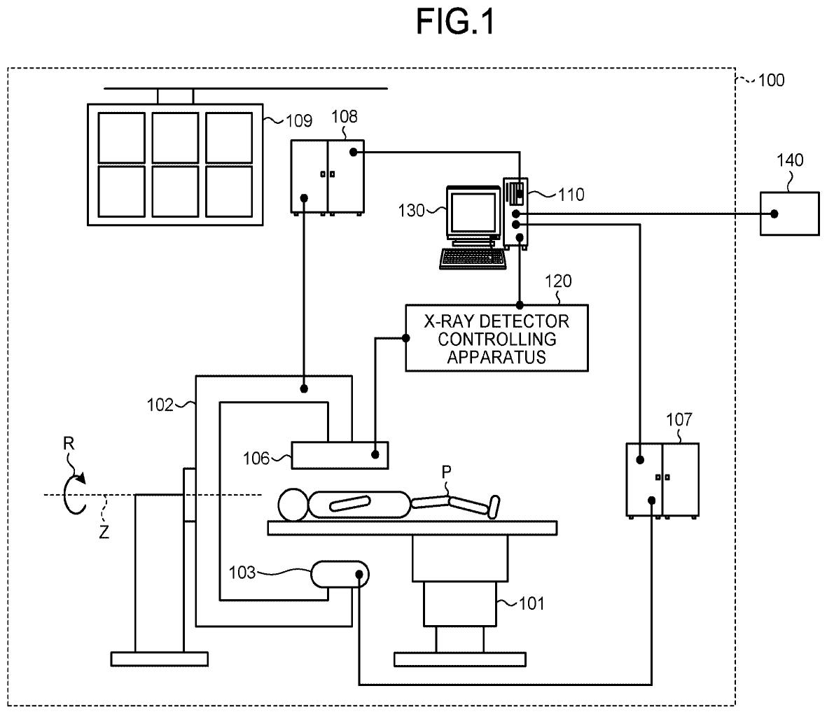

FIG. 1 is a block diagram illustrating an exemplary configuration of an X-ray diagnosis apparatus 100 according to an embodiment. As illustrated in FIG. 1, the X-ray diagnosis apparatus 100 according to the embodiment includes a catheter table 101, a holding apparatus 102, an X-ray tube 103, an X-ray detector 106, an X-ray high-voltage generating apparatus 107, a holding apparatus controlling apparatus 108, a monitor 109, a medical image processing apparatus 110, an X-ray detector controlling apparatus 120, and an input interface 130. The X-ray diagnosis apparatus 100 illustrated in FIG. 1 may be referred to as an X-ray cardiovascular diagnosis apparatus.

The catheter table 101 includes a tabletop and a base. The tabletop has an examined subject (hereinafter, “patient”) P placed thereon. For example, the base is configured to support the tabletop so as to be able to make parallel movements along the long-axis directions of the tabletop, the short-axis directions of the tabletop, and the vertical directions or the like. Further, the base is configured to rotatably support the tabletop, while using, as rotation axes, the short-axis direction of the tabletop, the long-axis direction of the tabletop, and the vertical direction or the like. A processor installed in the catheter table 101 is configured, under control of the medical image processing apparatus 110, to control various types of moving operations of the tabletop, on the basis of input operations received from an operator via an operating unit provided for the tabletop or the like, for example.

The holding apparatus 102 is capable of rotating on a Z-axis in the direction indicated with the arrow R and is configured to hold the X-ray tube 103 and the X-ray detector 106 so as to oppose each other. The holding apparatus 102 is configured to rotatably support an X-ray limiter attached to an X-ray emission window of the X-ray tube 103 and the X-ray detector 106, while using, as a rotation axis, a straight line connecting a focal point at which X-rays are generated by the X-ray tube 103, to a central part of the X-ray detector 106. As a result of operations of a holding arm moving mechanism, the holding apparatus 102 is configured to rotate the X-ray limiter and the X-ray detector 106 on the rotation axis.

In this situation, the holding apparatus 102 may be configured to hold the X-ray tube 103 and the X-ray detector 106 while a Source Image Distance (hereinafter, “SID”) is variable, for example. Possible shapes and configurations of the holding apparatus 102 are not limited to those illustrated in FIG. 1. For example, the holding apparatus 102 may be hung by an Ω-arm from the ceiling of an examination room in which the X-ray diagnosis apparatus 100 is provided. Alternatively, the holding apparatus 102 may be realized by using two arms such as a C-arm installed on the floor surface of the examination room and an Ω-arm hung from the ceiling. It is possible to use any of other known holding apparatuses, as appropriate.

The X-ray tube 103 is a vacuum tube configured to generate the X-rays by causing thermo electrons to be emitted from a negative pole (a filament) toward a positive pole (a target or an anode), with application of high voltage and a supply of a filament current from the X-ray high-voltage generating apparatus 107. As a result of the thermo electrons colliding with the target, the X-ray tube 103 is configured to generate the X-rays. For instance, examples of the X-ray tube 103 include an X-ray tube of an anode rotating type configured to generate the X-rays by having the thermo electrons emitted onto a rotating anode. However, applicable types of the X-ray tube 103 are not limited to the rotating anode type. It is acceptable to use an X-ray tube of an arbitrary type. Further, the X-ray emission window of the X-ray tube 103 is provided with the X-ray limiter and a radiation quality adjusting filter (which may be referred to as a collimator). The X-ray limiter and the radiation quality adjusting filter are used for the purpose of reducing radiation exposure of the patient P and enhancing image quality of image data.

The X-ray detector 106 is configured to detect X-rays that were emitted from the X-ray tube 103 and have passed through the patient P. For example, the X-ray detector 106 is realized by using a Flat Panel Detector (hereinafter, “FPD”). The FPD includes a plurality of semiconductor detecting elements. Examples of the semiconductor detecting elements include those of a direct conversion type configured to directly convert the X-rays into an electrical signal and those of an indirect conversion type configured to convert the X-rays into light via fluorescence and to further convert the light into an electrical signal. The FPD may be of either type. The electrical signal generated by the FPD is output to the medical image processing apparatus 110 via the X-ray detector controlling apparatus 120. Possible examples of the X-ray detector 106 are not limited to FPDs, and it is acceptable to use a detector having any other known configurations, as appropriate.

The X-ray high-voltage generating apparatus 107 includes, for example, electrical circuits such as a transformer and a rectifier, as well as a high-voltage generating unit. Under the control of the medical image processing apparatus 110, the high-voltage generating unit is configured to control output voltage corresponding to the X-rays to be emitted by the X-ray tube 103. As a result, the high-voltage generating unit has a function of generating the high voltage to be applied to the X-ray tube 103 and the filament current to be supplied to the X-ray tube 103. The X-ray high-voltage generating apparatus 107 may be of a transformer type or may be of an inverter type. Further, the X-ray high-voltage generating apparatus 107 may be provided for the holding apparatus 102.

Under the control of the medical image processing apparatus 110, the holding apparatus controlling apparatus 108 is configured to control the rotation of the holding apparatus 102 and the like. For example, the holding apparatus controlling apparatus 108 is configured to control various types of moving operations of the holding apparatus 102, on the basis of input operations received from the operator via an operating unit provided for the tabletop or the like.

The monitor (a display unit) 109 is configured to display X-ray images generated by the medical image processing apparatus 110, and the like. The monitor 109 may be configured by using a plurality of sub-monitors or may be a large-screen monitor of which the display region can arbitrarily be divided into sections in accordance with an instruction from the operator. Further, when the monitor 109 has the plurality of sub-monitors, the display region of any of the sub-monitors may arbitrarily be divided into sections in accordance with an instruction from the operator. Furthermore, the monitor 109 may be realized as a display. In that situation, as the display, for example, it is possible to use a Liquid Crystal Display (LCD), a Cathode Ray Tube (CRT) display, an Organic Electroluminescence Display (OELD) device, a plasma display, or other arbitrary displays, as appropriate. Further, the display may be of a desktop type or may be configured by using a tablet terminal or the like capable of wirelessly communicating with the medical image processing apparatus 110 and the like.

The medical image processing apparatus 110 is configured to control the holding apparatus controlling apparatus 108 and the X-ray high-voltage generating apparatus 107, to acquire the image data output by the X-ray detector controlling apparatus 120, and to perform image processing processes thereon. Details of the medical image processing apparatus 110 will be explained later.

The X-ray detector controlling apparatus 120 is configured to control timing for reading the electrical signal output by the X-ray detector 106. Further, the X-ray detector controlling apparatus 120 is configured to acquire the electrical signal from the X-ray detector 106, to generate the image data from the acquired electrical signal, and to output the image data to the medical image processing apparatus 110. For example, the X-ray detector controlling apparatus 120 is structured by using a charge/voltage converter, an Analog-to-Digital (A/D) converter, a parallel/serial converter, a gate driver, and the like. The charge/voltage converter is configured to convert the electrical signal output from the X-ray detector 106 into a voltage signal and to output the voltage signal to the A/D converter. Under the control of the medical image processing apparatus 110, the A/D converter is configured to convert the voltage signal converted from the electrical signal into X-ray image data realized as digital data and to output the X-ray image data to the parallel/serial converter. Under the control of the medical image processing apparatus 110, the parallel/serial converter is configured to convert the X-ray image data resulting from the A/D conversion from parallel data into serial data and to output the serial data to the medical image processing apparatus 110. Under the control of the medical image processing apparatus 110, the gate driver is configured to drive the detecting elements in the X-ray detector 106.

The input interface 130 is a keyboard, a control panel, a foot switch, and/or the like and is configured to receive inputs of various types of operations performed on the X-ray diagnosis apparatus 100 from the operator. In the present embodiment, the input interface 130 does not necessarily need to include physical operation component parts such as a mouse, a keyboard, a trackball, a switch, a button, a joystick, a touchpad, a touch panel display, and/or the like. For instance, possible examples of the input interface 130 include an electrical signal processing circuitry configured to receive an electrical signal corresponding to an input operation from an external input device provided separately from the apparatus and to output the electrical signal to the medical image processing apparatus 110 or the like. Further, the input interface 130 may be configured by using a tablet terminal or the like capable of wirelessly communicating with the medical image processing apparatus 110 and the like.

A biological information detecting apparatus 140 is configured to detect biological information of the patient P. For example, when the biological information is an electrocardiographic waveform, the biological information detecting apparatus 140 corresponds to an electrocardiograph. In this situation, the biological information detecting apparatus 140 is configured to output the electrocardiographic waveform of the patient P to the medical image processing apparatus 110. Further, the biological information does not necessarily need to be the electrocardiographic waveform and may be a respiratory waveform, a pulse waveform, or the like, for example. In those situations, the biological information detecting apparatus 140 corresponds to a respiratory waveform detector, a plethysmograph, or the like. Further, the biological information detecting apparatus 140 does not necessarily have to be a single measuring apparatus and may include an electrocardiograph and a respiratory waveform detector, for example. In that situation, the electrocardiograph is configured to output the electrocardiographic waveform of the patient P to the medical image processing apparatus 110, whereas the respiratory waveform detector is configured to output the respiratory waveform of the patient P to the medical image processing apparatus 110. Because it is possible to use a known apparatus as the biological information detecting apparatus 140, explanations thereof will be omitted. Further, although the biological information detecting apparatus 140 is positioned apart from the patient P in FIG. 1, the biological information detecting apparatus 140 may be positioned adjacent to the patient P or in the vicinity of the patient P.

Processing circuitry realizing the holding apparatus controlling apparatus 108, the X-ray detector controlling apparatus 120, and the like includes, as hardware resources thereof, a processor such as a Central Processing Unit (CPU), a Micro Processing Unit (MPU), or a Graphics Processing Unit (GPU), and memory elements such as a Read-Only Memory (ROM), a Random Access Memory, and/or the like.

Various types of functions implemented by the processor are stored in a memory (not illustrated) in the form of computer-executable programs. The processor is a processor configured to realize the functions corresponding to the programs, by reading and executing the programs from the memory. In other words, a plurality of circuits that have read the programs have the functions corresponding to the read programs.

The processing circuit may be realized by using an Application Specific Integrated Circuit (ASIC), a Field Programmable Gate Array (FPGA), or other processors such as a Complex Programmable Logic Device (CPLD) or a Simple Programmable Logic Device (SPLD).

An overall configuration of the X-ray diagnosis apparatus 100 according to the embodiment has thus been explained. In this configuration, the X-ray diagnosis apparatus 100 according to the embodiment is configured to acquire an X-ray signal output by the X-ray detector 106. Subsequently, the X-ray diagnosis apparatus 100 is configured to cause the monitor 109 to display an image generated from the acquired X-ray signal.

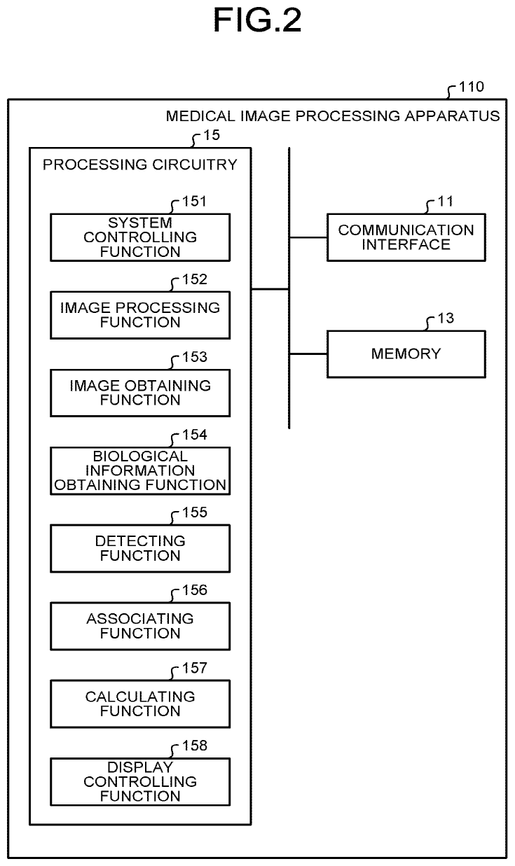

Next, a configuration of the medical image processing apparatus 110 will be explained, with reference to FIG. 2. FIG. 2 is a block diagram illustrating an exemplary configuration of the medical image processing apparatus 110. As illustrated in FIG. 2, the medical image processing apparatus 110 includes a communication interface 11, a memory 13, and processing circuitry 15. In the medical image processing apparatus 110, as illustrated in FIG. 2, the communication interface 11, the memory 13, and the processing circuitry 15 are electrically connected to one another via a bus.

The communication interface 11 is electrically connected to the catheter table 101, the holding apparatus 102, the X-ray high-voltage generating apparatus 107, the holding apparatus controlling apparatus 108, the monitor 109, the X-ray detector controlling apparatus 120, and the input interface 130. Further, the communication interface 11 is connected to a network. To the network, various types of modalities, a Hospital Information system (hereinafter, “HIS”), a medical image management system (hereinafter, “Picture Archiving and Communication System (PACS)”), and the like are connected.

The memory 13 is realized by using storage circuitry configured to store therein various types of information. For example, the memory 13 is a storage apparatus such as a Hard Disk Drive (HDD), a Solid State Drive (SSD), or an integration circuit storage apparatus. The memory 13 corresponds to a storage unit. Alternatively, other than being an HDD or an SSD, the memory 13 may be a semiconductor memory element such as a Random Access Memory (RAM) or a flash memory, an optical disc such as a Compact Disc (CD) or a Digital Versatile Disc (DVD), or a drive apparatus configured to read and write various types of information from and to a portable storage medium or a semiconductor memory element such as a RAM.

By employing an image obtaining function 153, the memory 13 is configured to store therein the X-ray images obtained from the X-ray detector controlling apparatus 120 via the communication interface 11. By employing a biological information obtaining function 154, the memory 13 is configured to store therein the biological information obtained from the biological information detecting apparatus 140 via the communication interface 11. For example, when an electrocardiographic waveform is obtained as the biological information, the memory 13 is configured, by employing an associating function 156, to store therein a cardiac phase at the time of an X-ray emission and an X-ray image generated as a result of the X-ray emission, so as to be kept in association with each other. As illustrated in FIG. 2, the memory 13 is configured to store therein the programs corresponding to the various types of functions implemented by the processing circuitry 15.

The memory 13 is configured to store therein a correspondence table (hereinafter, “device distance correspondence table”) indicating, with respect to the type of a device to be inserted in the patient P, a distance (hereinafter, “end face distance”) from a marker related to the device to an end face related to the device. The device may be a stent, for example. The marker related to the device corresponds to a marker provided for a balloon related to the device, for example. The marker is structured, for example, by using a substance having an X-ray attenuation coefficient larger than that of biological tissues, i.e., a high absorption material (any of various types of metal) having an X-ray absorption rate higher than that of biological tissues. Regarding the balloon and the stent set with a catheter, the end face distance corresponds to the distance from the marker on the balloon to the closest end face of the stent, for example.

Further, when a second stent is to be further placed after a first stent is placed, the memory 13 is configured to store therein a distance (hereinafter, “target distance”) from the position of an end face (hereinafter, “end face position”) of the first stent to a second marker related to the second stent, the distance including a length (overlapping amount information) by which the first stent and the second stent overlap with each other. The target distance is set in advance on the basis of the overlapping amount information, the end face distance, and a detected position of the marker (explained later). In other words, the target distance includes the distance related to the overlap between the first stent corresponding to a first device and the second stent corresponding to a second device.

On the basis of the electrical signals representing input operations and being output from the input interface 130, the processing circuitry 15 is configured to control the entirety of the X-ray diagnosis apparatus 100 and the medical image processing apparatus 110. For example, the processing circuitry 15 includes, as hardware resources thereof, a processor such as a CPU, an MPU, or a Graphics Processing Unit (GPU) and memory elements such as a ROM, a RAM, and/or the like.

Various types of processing functions implemented by the processing circuitry 15 are stored in the memory 13 in the form of computer-executable programs. The processing circuitry 15 is a processor configured to realize the various types of functions illustrated in FIG. 2 and corresponding to the programs, by reading and executing the programs from the memory 13. In other words, a plurality of circuits that have read the programs have the functions corresponding to the read programs.

The processing circuitry 15 includes, for example, a system controlling function 151, an image processing function 152, the image obtaining function 153, the biological information obtaining function 154, a detecting function 155, the associating function 156, a calculating function 157, and a display controlling function 158. In this situation, by employing the processor configured to execute the programs loaded into the memory, the processing circuitry 15 is configured to implement the system controlling function 151, the image processing function 152, the image obtaining function 153, the biological information obtaining function 154, the detecting function 155, the associating function 156, the calculating function 157, and the display controlling function 158. The processing circuitry 15 implementing the system controlling function 151, the image processing function 152, the image obtaining function 153, the biological information obtaining function 154, the detecting function 155, the associating function 156, the calculating function 157, and the display controlling function 158 corresponds to a system controlling unit, an image processing unit, an image obtaining unit, a biological information obtaining unit, a detecting unit, an associating unit, a calculating unit, and a display controlling unit.

In this situation, the system controlling function 151, the image processing function 152, the image obtaining function 153, the biological information obtaining function 154, the detecting function 155, the associating function 156, the calculating function 157, and the display controlling function 158 do not necessarily have to be realized by using the single processing circuit. It is also acceptable to structure a processing circuit by combining together a plurality of independent processors, so that the system controlling function 151, the image processing function 152, the image obtaining function 153, the biological information obtaining function 154, the detecting function 155, the associating function 156, the calculating function 157, and the display controlling function 158 are realized as a result of the processors executing the programs. Further, the processing circuitry 15 may be realized by using one or more processors each configured with an ASIC, an FPGA, a CPLD, an SPLD, or the like.

On the basis of the input operations received from the operator via the input interface 130, the processing circuitry 15 is configured, by employing the system controlling function 151, to control the X-ray high-voltage generating apparatus 107, the holding apparatus controlling apparatus 108, the monitor 109, the X-ray detector controlling apparatus 120, and the like. More specifically, the system controlling function 151 is configured to read a control program stored in the memory 13, to load the read control program into a memory in the processing circuitry 15, and to control functional units of the X-ray diagnosis apparatus 100 according to the loaded control program. Further, when the medical image processing apparatus 110 is realized as a stand-alone apparatus separate from modalities, the system controlling function 151 may be omitted, for example.

By employing the image processing function 152, the processing circuitry 15 is configured to receive an input signal output from the input interface 130 and to generate image data by performing various types of image processing processes such as a filtering process on the X-ray images. The image data corresponds to data of medical images including fluoroscopic images and/or taken images related to the patient P. The image processing function 152 is configured to perform a combining process, a subtraction process, and/or the like by using the image data. The processing circuitry 15 is configured to output the generated image data to the memory 13.

By employing the image obtaining function 153, the processing circuitry 15 is configured to obtain the X-ray images from the X-ray detector controlling apparatus 120, via the communication interface 11. For example, the image obtaining function 153 is configured to obtain a plurality of first X-ray images related to the patient P and a second X-ray image related to the patient P taken later than the first X-ray images. For example, the first X-ray images correspond to a plurality of fluoroscopic images displayed on the monitor 109 at the time of placing the first stent for the patient P. Further, for example, the second X-ray image corresponds to a plurality of fluoroscopic images displayed on the monitor 109, at the time of placing the second stent for the patient P after the first stent is placed for the patient P.

Further, the image obtaining function 153 may have a function of acquiring the electrical signal from the X-ray detector 106 and generating the image data (the X-ray images) from the acquired electrical signal. In other words, the image obtaining function 153 may be configured to generate (obtain) the X-ray images on the basis of the output from the X-ray detector 106. In that situation, the X-ray detector controlling apparatus 120 has only the function of controlling the timing for reading the electrical signal output by the X-ray detector 106.

By employing the biological information obtaining function 154, the processing circuitry 15 is configured to obtain the biological information from the biological information detecting apparatus 140 via the communication interface 11. For example, the biological information obtaining function 154 is configured to obtain first biological information related to periodic movements of the patient P at the times of taking the plurality of first X-ray images. The periodic movements of the patient P correspond, more specifically, to periodic movements of a predetermined body site of the patient. The predetermined body site may be the heart, the lungs, or the like, for example. Further, the biological information obtaining function 154 is configured to obtain second biological information related to a periodic movement of the patient P at the time of taking the second X-ray image. In the following sections, to explain a specific example, it is assumed that the first biological information and the second biological information are electrocardiographic waveforms of the patient P. Further, the image obtaining function 153 and the biological information obtaining function 154 may be realized as an integrated obtaining function.

From each of the plurality of first X-ray images, the processing circuitry 15 is configured, by employing the detecting function 155, to detect the position of a characteristic part related to the first device. The characteristic part is, for example, markers (hereinafter, “first markers”) provided for a balloon (hereinafter, “first balloon”) related to the first device. From each of the plurality of first X-ray images, the detecting function 155 is configured to detect the first markers by performing a threshold value judging process using pixel values and/or any of various types of segmentation processes.

In this situation, possible methods for detecting the first markers are not limited to the threshold value judging process using the pixel values and the various types of segmentation processes, and it is possible to use any of known image processing techniques as appropriate. Alternatively, the processes performed by the detecting function 155 may be realized by the image processing function 152 instead.

By employing the associating function 156, the processing circuitry 15 is configured to associate the position of the characteristic part with time phases in the first biological information, on the basis of the plurality of first X-ray images and the first biological information. With respect to each of the plurality of first X-ray images, the associating function 156 is configured to store, into the memory 13, the position of the characteristic part kept in association with the time phase.

By employing the calculating function 157, the processing circuitry 15 is configured to calculate a placement position of the second device in the time phases in the first biological information, on the basis of a distance related to an overlap between the first device and the second device in the second X-ray image and of device information. For example, the device information denotes one or both of the position of an end face of the first stent and the positions of the first markers. More specifically, the calculating function 157 is configured to determine an end face distance of the first device (hereinafter, “first end face distance”), on the basis of the type of the first device and the device distance correspondence table. Subsequently, in each of the plurality of first X-ray images, the calculating function 157 is configured to calculate, on the basis of the positions of the first markers and the first end face distance, the positions of the end faces of the first device, i.e., the positions of the end faces of the first stent (hereinafter, “end face positions”).

On the basis of the distance related to the overlap between the first device and the second device and of the device information, the calculating function 157 is configured to calculate placement positions of the second device in the time phases in the first biological information. More specifically, on the basis of the target distance and the first markers, the calculating function 157 is configured to calculate a placement position in which the second stent is to be placed, with respect to each of the plurality of time phases corresponding to the plurality of first X-ray images. For example, the placement positions each correspond to the position of the second marker, in an overlap region between the first stent and the second balloon. In other words, the placement positions each indicate a recommended position (hereinafter, “target end face”) to be reached by the second marker in an insertion direction of the second device toward a site of interest such as a blood vessel stenosis site. The target end face may be recommended as a recommended position.

By employing the display controlling function 158, the processing circuitry 15 is configured to cause the monitor (the display) 109 to display the device information related to the first device so as to be superimposed on the second X-ray image, by using the result of the associating process performed by the associating function 156 and the time phase in the second biological information. The display controlling function 158 is configured to cause the monitor (the display) 109 to display the placement position of the second stent (i.e., target end face) so as to be further superimposed on the second X-ray image.

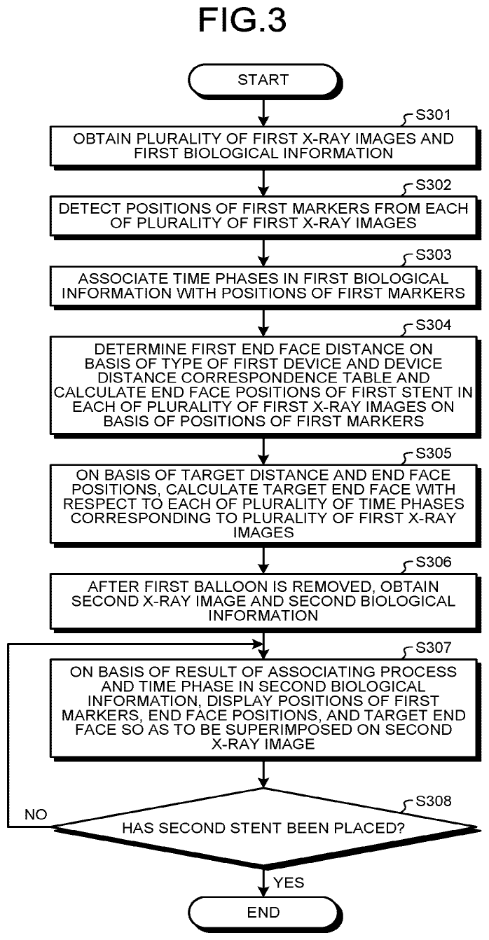

A configuration of the medical image processing apparatus 110 according to the embodiment has thus been explained. In this configuration, the X-ray diagnosis apparatus 100 and the medical image processing apparatus 110 according to the embodiment are configured to perform the process (hereinafter, “device display process”) of causing the device information related to the placed first stent to be displayed over the second X-ray image. In the following sections, a procedure in the device display process will be explained with reference to FIG. 3. FIG. 3 is a flowchart illustrating an example of the procedure in the device display process.

### The Device Display Process

### Step S301:

The image obtaining function 153 obtains the plurality of first X-ray images by carrying out an imaging process on the patient P. More specifically, the image obtaining function 153 obtains the plurality of first X-ray images in a time series. For example, the plurality of first X-ray images are acquired over a predetermined time period. The predetermined time period may be, for example, a time period corresponding to one cardiac cycle. In addition, by employing the biological information obtaining function 154, the processing circuitry 15 obtains an electrocardiographic waveform as the first biological information corresponding to the plurality of first X-ray images. Further, by employing the associating function 156, the processing circuitry 15 associates each of the plurality of first X-ray images with a cardiac phase, on the basis of X-ray emission timing related to generating the plurality of first X-ray images and the cardiac phases in the electrocardiographic waveform at the emission timing.

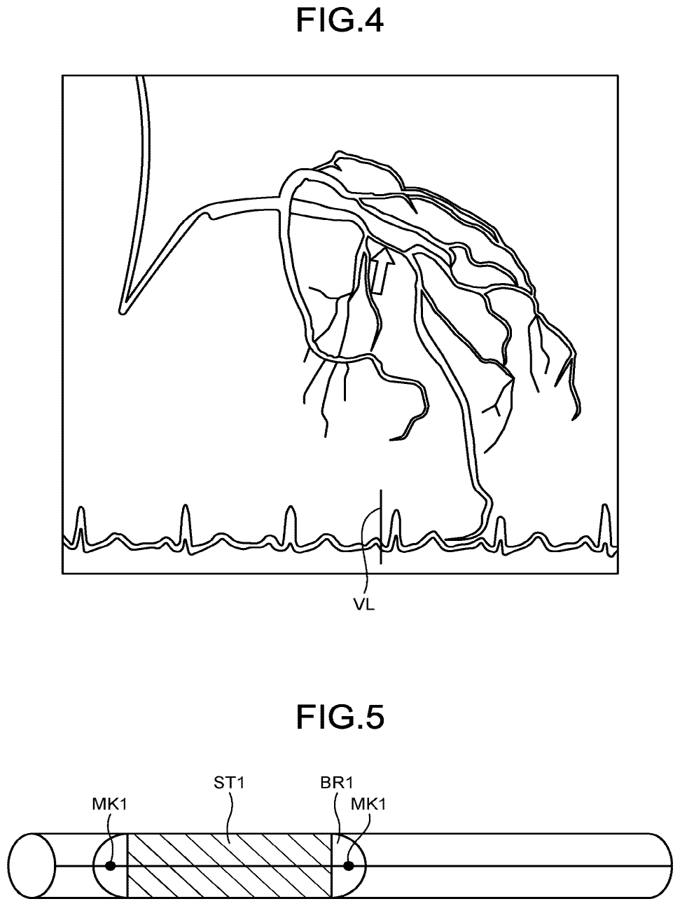

FIG. 4 is a drawing illustrating an example of an X-ray image (a fluoroscopic image) before obtaining the plurality of first X-ray images. The arrow in FIG. 4 indicates a blood vessel stenosis site of the patient P. The first device is to be moved to the blood vessel stenosis site. Displayed at the bottom the X-ray image in FIG. 4 is the electrocardiographic waveform serving as biological of the patient P. The vertical line VL in the electrocardiographic waveform indicates the cardiac phase corresponding to the X-ray image presented in FIG. 4.

### Step S302:

By employing the detecting function 155, the processing circuitry 15 detects the positions of the first markers, with respect to each of the plurality of first X-ray images. In this situation, when there is a frame (hereinafter, “undetectable frame”) from which the positions of the first markers are undetectable among the plurality of first X-ray images (a plurality of frames) in one cycle, the processing circuitry 15 may further obtain a plurality of first X-ray images by extending the predetermined time period while employing the image obtaining function 153. Further, the predetermined time period may be set so as to correspond to a plurality of cardiac cycles, in consideration of occurrence of random image noise in the first X-ray images. With this arrangement, it is possible to reduce frequency with which the positions of the first markers are undetectable due to impact of the image noise.

Alternatively, the detecting function 155 may detect the positions of the first markers with respect to each of a plurality of first X-ray images related to a specific cardiac phase during the cardiac cycle. For example, the specific cardiac phase may be an R-wave, a T-wave, a S-wave, or the like in the electrocardiographic waveform and is set in advance. Further, when there is an undetectable frame, the detecting function 155 may determine the positions of the first markers in the undetectable frame, by performing an interpolation process using at least two sets of positions of the first markers in certain frames (hereinafter, “detectable frames”) which are close to the undetectable frame and from which the positions of the first markers were detected and/or an interpolation process using positions of the first markers corresponding to a plurality of cycles.

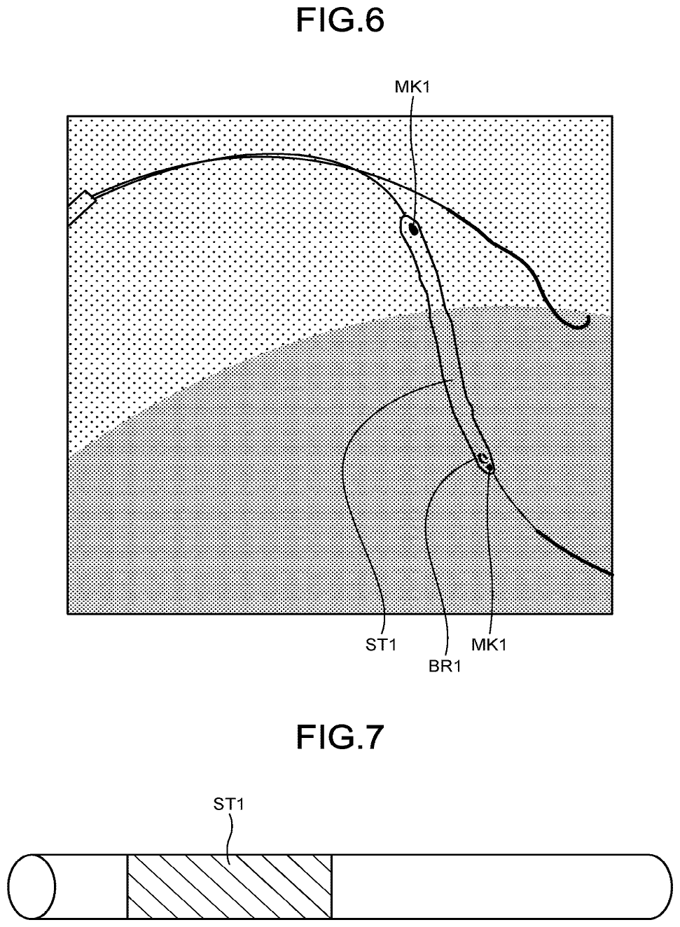

FIGS. 5 and 6 are drawings illustrating display examples in a frame at the time of an expansion of a first balloon BR1 moved to the blood vessel stenosis site. As illustrated in FIGS. 5 and 6, first markers MK1 are provided in the vicinity of the two ends of the first balloon BR1. Further, as illustrated in FIGS. 5 and 6, as the first balloon BR1 expands, a first stent ST1 has the shape of the expanded stenosis site.

In this situation, in response to detection of the expansion of the first balloon BR1 (hereinafter, “balloon expansion detection”), the processing circuitry 15 may be configured to cause the detecting function 155 to start detecting the positions of the first markers MK1 from the first X-ray image in the frame corresponding to the balloon expansion detection. In that situation, the start of the detection of the positions of the first markers MK1 are automated. The balloon expansion detection is implemented by the image processing function 152, for example, by calculating the differences in the X-ray images among the frames and/or performing any of various types of segmentation processes.

FIG. 7 is a drawing illustrating an example after the first balloon BR1 is removed subsequent to the expansion of the first balloon BR1. As illustrated in FIG. 7, the first stent ST1 is placed in the expanded stenosis site.

### Step S303:

By employing the associating function 156, the processing circuitry 15 associates the time phases in the first biological information with the positions of the first markers MK1. For example, with respect to each of the plurality of first X-ray images, the associating function 156 associates the time phase in the electrocardiographic waveform at the time of obtaining the first X-ray image, with the positions of the first markers MK1. The positions of the first markers MK1 kept in association with the time phases correspond to virtual positions of the first markers and are stored into the memory 13. As a result, the memory 13 has recorded therein to which the positions the first markers MK1 are to move, due to pulsation of the heart of the patient P.

### Step S304:

By employing the calculating function 157, the processing circuitry 15 determines the first end face distance on the basis of the type of the first device and the device distance correspondence table. Subsequently, the calculating function 157 calculates, with respect to each of the plurality of first X-ray images, the end face positions of the first stent ST1, on the basis of the positions of the first markers MK1 and the first end face distance.

By employing the associating function 156, the processing circuitry 15 associates the time phases related to the positions of the first markers MK1, with the calculated end face positions. The end face positions kept in association with the time phases correspond to virtual end face positions related to the first stent ST1 and are stored in the memory 13. As a result, the memory 13 has recorded therein to which positions the end face positions are to move, due to pulsation of the heart of the patient P.

### Step S305:

By employing the calculating function 157, the processing circuitry 15 calculates, with respect to each of the plurality of time phases corresponding to the plurality of first X-ray images, the target end face on the basis of the target distance and the end face positions. For example, the target end face is at the location reached by advancing by the target distance from one of the end face positions of the first stent ST1 toward the other end face position of the first stent ST1. In other words, the target end face is calculated so that the first stent ST1 overlaps with the second stent when the second marker has reached the target end face.

### Step S306:

The image obtaining function 153 obtains the second X-ray image related to the patient P taken later than the plurality of first X-ray images. More specifically, the image obtaining function 153 obtains the second X-ray image by imaging the patient P after the first stent ST1 is placed. In addition, by employing the biological information obtaining function 154, the processing circuitry 15 obtains an electrocardiographic waveform as the second biological information corresponding to the second X-ray image. Furthermore, by employing the associating function 156, the processing circuitry 15 associates the second X-ray image with a cardiac phase, on the basis of the X-ray emission timing related to generating the second X-ray image and the cardiac phase in the electrocardiographic waveform corresponding to the emission timing. In this situation, the second device is inserted in the patient P, and the second marker is moved by a user's operation.

FIG. 7 is a drawing illustrating an example of the first stent ST1 after the first balloon BR1 is removed. Although the first stent ST1 is clearly indicated in FIG. 7, in many situations, the placed stent may not be clearly visible.

### Step S307:

By employing the display controlling function 158, the processing circuitry 15 causes the display 109 to display the device information related to the first device so as to be superimposed on the second X-ray image, by using the result of the associating process performed by the associating function 156 and the time phase in the second biological information. More specifically, while using the time phase in the electrocardiographic waveform kept in association with the second X-ray image as an input, the display controlling function 158 identifies the positions of the first markers MK1, the end face positions, and the placement position related to the time phase in the electrocardiographic waveform kept in association with the second X-ray image, via the correspondence relationship between the time phases in the electrocardiographic waveform related to the first biological information and the positions of the first markers MK1. The display controlling function 158 causes the monitor 109 to display at least one selected from among the positions of the first markers MK1, the end face positions, and the placement position (the target end face) that were identified, so as to be superimposed on the second X-ray image.

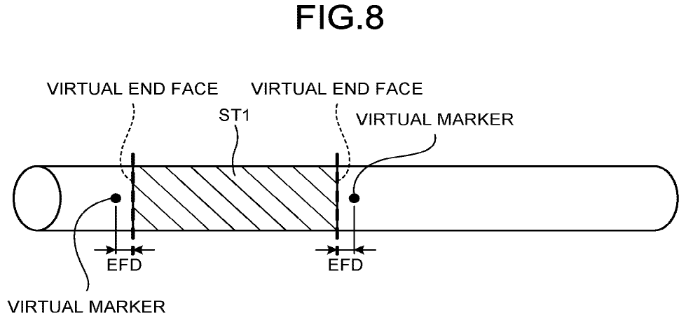

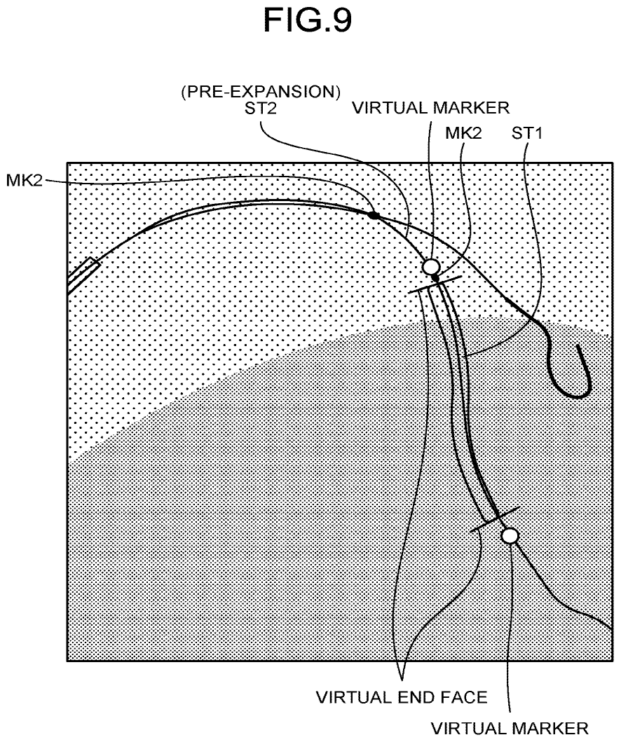

FIGS. 8 and 9 are drawings each illustrating examples of the positions of the first markers MK1 (the virtual markers) and the end face positions (the virtual end faces) of the first stent ST1 superimposed on a blood vessel region in the second X-ray image. As illustrated in FIGS. 8 and 9, the virtual markers and the virtual end faces are superimposed on the second X-ray image by using a display mode that is visually recognizable by the user. In FIG. 8, “EFD” denotes the end face distance. In FIG. 9, the second stent ST2 is in a pre-expansion state.

### Step S308:

When the second stent ST2 has been placed (step S308: Yes), the device display process ends. In other words, after the second marker is moved to the placement position, the second balloon is expanded. At this time, the second stent ST2 is expanded and placed, and the device display process thus ends. Alternatively, after the second stent ST2 is placed, the process at step S307 may be performed for a predetermined time period, before the device display process ends. On the contrary, when the second stent ST2 has not been placed at the placement position (step S308: No), the process at step S307 is repeatedly performed.

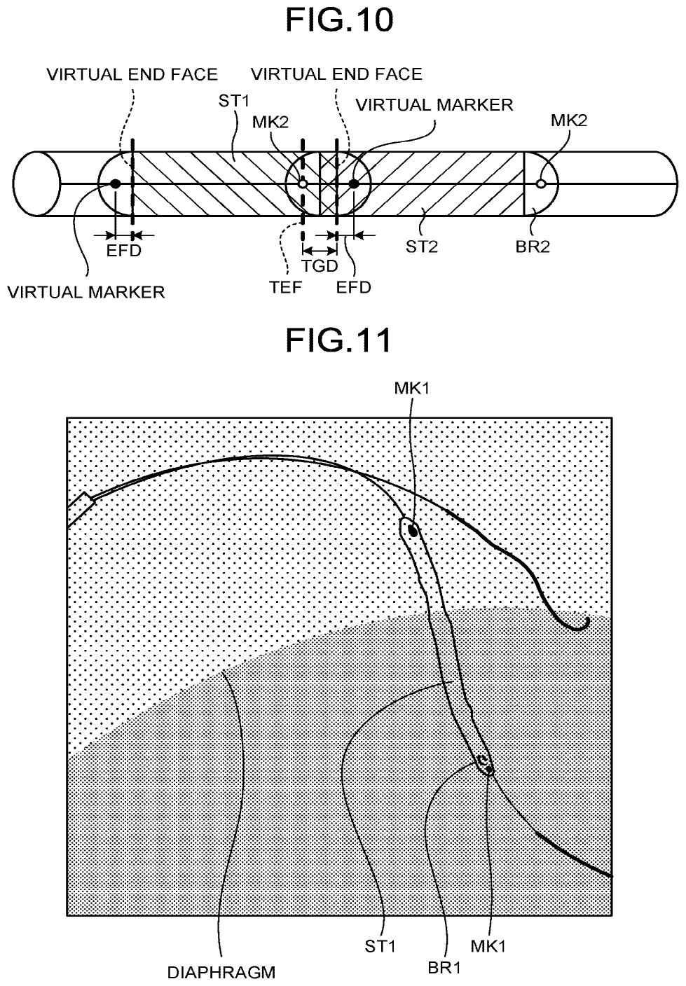

FIG. 10 is a drawing illustrating an example of a second marker MK2 moved up to a target end face TEF in a stenosis site. As illustrated in FIG. 10, the marker MK2 of the second balloon BR2 has been moved up to the target end face TEF serving as the placement position. In this situation, in conjunction with the expansion of the second balloon BR2, the second stent ST2 is expanded. In addition, as illustrated in FIG. 10, an end part of the first stent ST1 overlaps with an end part of the second stent ST2.

The X-ray diagnosis apparatus 100 according to the embodiment described above is configured: to obtain the plurality of first X-ray images related to the patient P and the second X-ray image related to the patient P taken later than the plurality of first X-ray images; to obtain the first biological information related to the periodic movements of the patient P at the times of taking the plurality of first X-ray images and the second biological information related to the periodic movement of the patient P at the time of taking the second X-ray image; to detect the position of the characteristic part related to the first device from each of the plurality of first X-ray images; to associate the position of the characteristic part with the time phases in the first biological information, on the basis of the plurality of first X-ray images and the first biological information; and to cause the display 109 to display the device information related to the first device so as to be superimposed on the second X-ray image, by using the result of the associating process and the time phase in the second biological information. In this situation, the first device is the first stent ST1. The characteristic part is the first markers MK1 provided for the first balloon BR1 related to the first stent ST1. The device information includes one or both of the positions of the end faces (the virtual end faces) of the first stent ST1 and the positions of the first markers MK1 (the virtual markers).

In other words, the X-ray diagnosis apparatus 100 in the present example is configured to associate the positions of the first markers MK1 detected from the first X-ray images as the virtual markers, with the time phases in a biological signal and to further determine, with respect to each of the time phases in the biological signal, the end face positions as the virtual end faces, on the basis of the positions of the first markers MK1, the type of the first device, and the device distance correspondence table. Subsequently, the X-ray diagnosis apparatus 100 is configured to determine the positions of the first markers MK1 and the end face positions corresponding to the time phase in a second biological signal in the second X-ray image and to cause the monitor 109 to display the positions of the first markers MK1 and the end face positions that were determined, so as to be superimposed on the second X-ray image.

With these configurations, by using the X-ray diagnosis apparatus 100, as illustrated in FIGS. 8 and 9, it is possible to enhance visibility, during the manipulation of placing the plurality of stents, of the information (the device information) related to the treatment device (e.g., the first stent ST1) in the X-ray images, after the first stent ST1 is placed.

Further, the X-ray diagnosis apparatus 100 is configured to calculate the placement position of the second device in a time phase in a first biological signal, on the basis of the distance related to the overlap between the first device and the second device in the second X-ray image and of the device information. The second device corresponds to the second stent ST2. In other words, on the basis of a target distance TGD and the first markers MK1, the X-ray diagnosis apparatus 100 is configured to calculate, with respect to each of the plurality of time phases corresponding to the plurality of first X-ray images, the placement position (the target end face TEF) in which the second stent ST2 is to be placed. Subsequently, the X-ray diagnosis apparatus 100 is configured to determine the placement position corresponding to the time phase in the second biological signal in the second X-ray image and to cause the monitor 109 to display the determined placement position so as to be superimposed on the second X-ray image.

As a result, as illustrated in FIG. 10 for example, the X-ray diagnosis apparatus 100 is configured, during the manipulation to place the plurality of stents, to cause the monitor 109 to display the placement position TEF of the second stent ST2 so as to be further superimposed on the second X-ray image, after the first stent ST1 is placed. With this configuration, by using the X-ray diagnosis apparatus 100, it is possible to place the second stent ST2 safely and in an appropriate position, i.e., in such a manner that an end part of the first stent ST1 appropriately overlaps with an end part of the second stent ST2.

Accordingly, the X-ray diagnosis apparatus 100 is able to provide the user with assistance related to the placement of the second stent ST2. Consequently, the X-ray diagnosis apparatus 100 makes it possible to place the second and later stents safely in appropriate positions, which thus realizes shortening treatment time and improving prognoses.

### First Modification Example

In the present modification example, an anatomical landmark in the first X-ray images is brought into association with the positions of the first markers MK1 superimposed on the second X-ray image. By employing the detecting function 155, the processing circuitry 15 is configured to detect a first anatomical landmark of the patient P from each of the plurality of first X-ray images. Further, the detecting function 155 is configured to detect a second anatomical landmark of the patient P from the second X-ray image.

For example, the first anatomical landmark and the second anatomical landmark may be the diaphragm and the vertebral body. To the process of detecting the anatomical landmarks from the X-ray images, because it is possible to apply a known process such as any of various types of segmentation processes and various types of Deep Neural Networks (DNNs) for image recognition (semantic segmentations), explanations thereof will be omitted.

FIG. 11 is a drawing illustrating an example in which the diaphragm is displayed in a first X-ray image. As illustrated in FIG. 11, for example, the detecting function 155 is configured to detect the diaphragm from the first X-ray image. The first anatomical landmark and the second anatomical landmark correspond to landmarks of mutually the same type.

By employing the associating function 156, the processing circuitry 15 is configured to further associate the first anatomical landmark with the position of a characteristic part. More specifically, the associating function 156 is configured to associate the positional relationship between the anatomical landmark and the first markers MK1, with the time phases in the electrocardiographic waveform of the first X-ray images.

By employing the display controlling function 158, the processing circuitry 15 is configured to cause the display 109 to display the device information related to the first device so as to be superimposed on the second X-ray image, by further using the first anatomical landmark and the second anatomical landmark. In other words, the display controlling function 158 is configured to determine the positions of the first markers MK1 in the second X-ray image, by performing a position alignment process on the second anatomical landmark in the second X-ray image and the first anatomical landmark. Subsequently, the display controlling function 158 is configured to cause the monitor 109 to display the determined positions of the first markers MK1 so as to be superimposed on the second X-ray image. As the positions of the first markers MK1 have been determined, the display controlling function 158 is configured to cause the monitor 109 to display the end face positions (the virtual end faces) and the target end face TEF so as to be superimposed on the second X-ray image. Alternatively, the abovementioned position alignment process may be performed by the image processing function 152 instead.

The X-ray diagnosis apparatus 100 according to the present modification example is configured to detect the first anatomical landmark of the patient P from each of the plurality of first X-ray images, to detect the second anatomical landmark of the patient P from the second X-ray image, to further associate the first anatomical landmark with the position of the characteristic part, and to cause the display 109 to display the device information related to the first device so as to be superimposed on the second X-ray image, by further using the first anatomical landmark and the second anatomical landmark. As a result, the X-ray diagnosis apparatus 100 is able to enhance visibility of the information related to the treatment device (e.g., the first stent ST1) in the X-ray images, even when, after the first stent ST1 is placed, another stent is to be placed so as to be joined with the first stent ST1 on a later day or the like. Because the other advantageous effects are the same as those of the embodiment, explanations thereof will be omitted.

### Second Modification Example

In a second modification example, on the basis of the display position of the device information in the second X-ray image, the display 109 is caused to display the second X-ray image on which the device information is superimposed, while the display position is fixed. More specifically, by employing the display controlling function 158, the processing circuitry 15 is configured to identify the display positions of the first markers MK1 in the second X-ray image. Subsequently, the display controlling function 158 is configured to cause the display 109 to display the second X-ray image on which the first markers MK1 are superimposed, while keeping the display positions of the first markers MK1 fixed. In this situation, instead of the positions of the first markers MK1 (the virtual markers), the display controlling function 158 may be configured to cause the display 109 to display the second X-ray image on which the end face positions (the virtual end faces) or the placement position (the target end face) are superimposed, while keeping the display positions of the end face positions or the placement position fixed. With this configuration, the X-ray diagnosis apparatus 100 according to the present modification example is able to display the device information, while keeping the display position of the device information fixed, such as the positions of the first markers MK1, the end face positions, or the placement position, in each of a plurality of second X-ray images that are displayed before the second stent ST2 is placed.

In other words, in the present modification example, it is possible to realize a function (a device stabilizing function) by which the device is displayed in a stable (fixed) manner in relation to the placement of the second stent ST2. With these configurations, it is possible to enhance visibility of the information related to the device information (e.g., the placement position of the second stent ST2) related to the first stent ST1 in the second X-ray image. In addition, according to the present modification example, as the second stent ST2 approaches the first stent ST1, because the movements of the second stent ST2 due to pulsation and/or respiration of the patient P become smaller, it is possible to enhance the assistance for the user related to the placement of the second stent ST2. Because the other advantageous effects are the same as those in the embodiment, explanations thereof will be omitted.

### Third Modification Example

In a third modification example, the display 109 is caused to display the second X-ray image, in such a manner that display contrast of a region different from a predetermined region including the device information is lower than display contrast of the predetermined region. The predetermined region may be, for example, a rectangular or circular region of a predetermined size including the positions of the first markers MK1, the end face positions, and the placement position TEF. More specifically, from the second X-ray image, the display controlling function 158 is configured to identify the other region excluding the predetermined region related to the device information including the positions of the first markers MK1, the end face positions, or the placement position. For example, the display controlling function 158 is configured to identify the other region by subtracting the predetermined region from the second X-ray image. Alternatively, the process of identifying the other region may be realized by the image processing function 152.

The display controlling function 158 is configured to cause the display 109 to display the second X-ray image in such a manner that the display contrast of the region different from the predetermined region including the device information is lower than the display contrast of the predetermined region. The X-ray diagnosis apparatus 100 according to the present modification example is able to further enhance the visibility of the information related to the device information (the positions of the first markers MK1, the end face positions, and the placement position) in the second X-ray image, after the first stent ST1 is placed. Because the other advantageous effects are the same as those of the embodiment, explanations thereof will be omitted.

When the technical concept of the embodiment is realized as a medical image processing program, the medical image processing program is configured to cause a computer to realize: obtaining the plurality of first X-ray images related to the patient P and the second X-ray image related to the patient P taken later than the plurality of first X-ray images; obtaining the first biological information related to the periodic movements of the patient P at the times of taking the plurality of first X-ray images and the second biological information related to the periodic movement of the patient P at the time of taking the second X-ray image; detecting the position of the characteristic part of the first device from each of the plurality of first X-ray images; associating the position of the characteristic part with the time phases in the first biological information, on the basis of the plurality of first X-ray images and the first biological information; and causing the display 109 to display the device information related to the first device so as to be superimposed on the second X-ray image, by using the result of the associating process and the time phase in the second biological information.

For example, it is also possible to realize the device display process by installing the medical image processing program in a computer of a medical image processing apparatus or the like and loading the program into a memory. In that situation, the program capable of causing the computer to execute the device display process may be distributed as being stored in a storage medium such as a magnetic disk (e.g., a hard disk), an optical disc (e.g., a Compact Disc Read-Only memory (CD-ROM) or a DVD), or a semiconductor memory. Because processing procedures and advantageous effects of the device display process program are the same as those of the embodiment, explanations thereof will be omitted.

According to at least one aspect of the embodiments and the like described above, it is possible to enhance the visibility of the information related to the treatment device in the X-ray images.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured:
to obtain a plurality of first X-ray images related to an examined subject and a second X-ray image related to the examined subject taken later than the plurality of first X-ray images;
to obtain first biological information related to a periodic movement of the examined subject at times of taking the plurality of first X-ray images and second biological information related to a periodic movement of the examined subject at a time of taking the second X-ray image;
to detect a position of a characteristic part related to a first device from each of the plurality of first X-ray images;
to associate the position of the characteristic part with a time phase in the first biological information, on a basis of the plurality of first X-ray images and the first biological information; and
to cause a display to display device information related to the first device so as to be superimposed on the second X-ray image, by using a result of the associating process and a time phase in the second biological information.

2. The medical image processing apparatus according to claim 1, wherein
the processing circuitry is configured to calculate a placement position of a second device in the time phase in the first biological information, on a basis of a distance related to an overlap between the first device and the second device in the second X-ray image and of the device information, and
the processing circuitry is configured to cause the display to display the placement position kept in association with the time phase in the second biological information, so as to be further superimposed on the second X-ray image.

3. The medical image processing apparatus according to claim 2, wherein
the first device is a first stent,
the second device is a second stent,
the characteristic part is a marker provided for a balloon related to the first stent, and
the device information includes one or both of a position of an end face of the first stent and a position of the marker.

4. The medical image processing apparatus according to claim 1, wherein
the processing circuitry is configured to detect a first anatomical landmark of the examined subject from each of the plurality of first X-ray images and to detect a second anatomical landmark of the examined subject from the second X-ray image,
the processing circuitry is configured to further associate the first anatomical landmark with the position of the characteristic part, and
the processing circuitry is configured to cause the display to display the device information related to the first device so as to be superimposed on the second X-ray image, by further using the first anatomical landmark and the second anatomical landmark.

5. The medical image processing apparatus according to claim 1, wherein, on a basis of a display position of the device information in the second X-ray image, the processing circuitry is configured to cause the display to display the second X-ray image on which the device information is superimposed, while keeping the display position fixed.

6. The medical image processing apparatus according to claim 1, wherein the processing circuitry is configured to cause the display to display the second X-ray image, in such a manner that display contrast of a region different from a predetermined region including the device information is lower than display contrast of the predetermined region.

7. An X-ray diagnosis apparatus comprising processing circuitry configured:
to obtain, by imaging an examined subject, a plurality of first X-ray images and a second X-ray image related to the examined subject taken later than the plurality of first X-ray images;
to obtain first biological information related to a periodic movement of the examined subject at times of taking the plurality of first X-ray images and second biological information related to a periodic movement of the examined subject at a time of taking the second X-ray image;
to detect a position of a characteristic part of a first device from each of the plurality of first X-ray images;
to associate the position of the characteristic part with a time phase in the first biological information, on a basis of the plurality of first X-ray images and the first biological information; and
to cause a display to display device information related to the first device so as to be superimposed on the second X-ray image, by using a result of the associating process and a time phase in the second biological information.

8. The X-ray diagnosis apparatus according to claim 7, wherein
the processing circuitry is configured to calculate a placement position of a second device in the time phase in the first biological information, on a basis of a distance related to an overlap between the first device and the second device in the second X-ray image and of the device information, and
the processing circuitry is configured to cause the display to display the placement position kept in association with the time phase in the second biological information, so as to be further superimposed on the second X-ray image.

9. The X-ray diagnosis apparatus according to claim 8, wherein
the first device is a first stent,
the second device is a second stent,
the characteristic part is a marker provided for a balloon related to the first stent, and
the device information includes one or both of a position of an end face of the first stent and a position of the marker.

10. The X-ray diagnosis apparatus according to claim 7, wherein
the processing circuitry is configured to detect a first anatomical landmark of the examined subject from each of the plurality of first X-ray images and to detect a second anatomical landmark of the examined subject from the second X-ray image,
the processing circuitry is configured to further associate the first anatomical landmark with the position of the characteristic part, and
the processing circuitry is configured to cause the display to display the device information related to the first device so as to be superimposed on the second X-ray image, by further using the first anatomical landmark and the second anatomical landmark.

11. The X-ray diagnosis apparatus according to claim 7, wherein, on a basis of a display position of the device information in the second X-ray image, the processing circuitry is configured to cause the display to display the second X-ray image on which the device information is superimposed, while keeping the display position fixed.

12. The X-ray diagnosis apparatus according to claim 7, wherein the processing circuitry is configured to cause the display to display the second X-ray image, in such a manner that display contrast of a region different from a predetermined region including the device information is lower than display contrast of the predetermined region.

13. A non-volatile computer-readable storage medium storing therein a medical image processing program that causes a computer to realize:
obtaining first biological information related to a periodic movement of an examined subject at times of taking a plurality of first X-ray images related to the examined subject and second biological information related to a periodic movement of the examined subject at a time of taking a second X-ray image related to the examined subject taken later than the plurality of first X-ray images;
detecting a position of a characteristic part of a first device from each of the plurality of first X-ray images;
associating the position of the characteristic part with a time phase in the first biological information, on a basis of the plurality of first X-ray images and the first biological information; and
causing a display to display device information related to the first device so as to be superimposed on the second X-ray image, by using a result of the associating process and a time phase in the second biological information.
